(19) United States
(12) Patent Application Publication
(10) Pub. No.: US 2007/0167693 A1
(43) Pub. Date: Jul. 19, 2007

(54) Display means for vital parameters

(76) Inventors: Bernd Scholler, Karlsruhe (DE); Thomas Magin, Schifferstadt (DE); Thomas Marx, Hamburg (DE); Klaus Forstner, Asperg (DE); Elmar Vitt, Rotenburg (DE); Carola Schmidt, Henstedt Ulzburg (DE)

Correspondence Address: Friedrich Kueffner, Suite 910, 317 Madison Avenue, New York, NY, 10017, US

(21) Appl. No.: 11/600,334

(22) Filed: Nov. 15, 2006

(30) Foreign Application Priority Data
Nov. 15, 2005 (DE) 10 2005 054 836.9

(51) Int. Cl. A61B 5/00; A61B 5/02; A61B 5/04; A61B 5/08

(52) U.S. Cl. 600/301; 600/485; 600/500; 600/549; 600/323; 600/529; 128/903; 600/509; 600/544; 600/546

(57) ABSTRACT

A display is connected to at least one sensor for detecting and transmitting physiological measurement values. In addition, one or more displays are used to present the measurement values and/or other associated data visually, acoustically, mechanically, or in some other way perceptible to the senses. A sequence control approach is implemented, according to which the measurement value, before reaching the display, passes through a testing stage, which, on the basis of at least one measured or stored additional value, verifies, checks, or changes the physiological measurement value detected at the sensor or supplements it with additional information. In a process for the noninvasive determination of the oxygen content CaO₂ of human and/or animal tissue, the blood oxygen saturation and data on the blood hemoglobin concentration are obtained after the measurements have been acquired by the use of radiation with wavelengths in the range of 400-1,800 nanometers.

### BACKGROUND OF THE INVENTION

1. Field of the Invention

The present invention relates to a display means with at least one sensor means for detecting and transmitting physiological measurement values and one or more display means for presenting the measurement values and/or other associated data visually, acoustically, mechanically, or by some other means perceptible to the senses.

The invention also relates to a process for the noninvasive determination of the oxygen content (CaO₂) of human and/or animal tissue, in which electromagnetic radiation from at least one source with wavelengths in the range of 400-1,800 nm is emitted; the radiation is conducted through the vessel/tissue; a detector detects the radiation after it has passed through the vessel/tissue and/or after it has been scattered by the vessel/tissue; and the data are digitized.

2. Description of the Related Art

The monitoring of individual body functions is known. For example, body functions such as heart rate, blood pressure, and body temperature are monitored. Multi-function units are also commercially available, which are worn like a wristwatch and display not only the blood pressure or pulse but also other measurement values such as the ambient temperature.

These types of display means, however, are limited to a just a few measurement variables and are therefore not really suitable for continuous monitoring when it is desired to know whether an important vital parameter such as the oxygen supply or the hemoglobin concentration of a user is in a critical range or not. In addition, the only prior art display means known so far are those which display a permanently preprogrammed set of measurement variables.

### SUMMARY OF THE INVENTION

It is desirable to design a display means in such a way that, without impairment to the well-being of its user, important vital parameters can be monitored so that warning of critical physical states can be given without the need for a physician to analyze the measurement data, where the user himself can decide which of the vital parameters are to be displayed and how they are to be displayed.

This object is accomplished according to the invention in that a sequence control approach is implemented, according to which the measurement value, before reaching the display, passes through a testing stage, which, on the basis of at least one measured or stored additional value, verifies, checks, or changes the physiological measurement value detected at the sensor or supplements it with additional information.

An additional object of the present invention is to improve a process of the type indicated above in such a way that that implementation of the process is optimized.

This object is accomplished according to the invention in that the blood oxygen saturation (SaO₂) can be determined by means of at least one mathematical link of the data; that the concentration of hemoglobin in the blood (cHb) can be determined by means of at least one other mathematical link of the data; and the saturations and/or concentrations of additional physiological measurements values can be determined by additional mathematical links.

According to the invention, in a display means

with at least one sensor arrangement with at least one first sensor means for the noninvasive detection of a vital body function;

with a sensor signal evaluation stage with a processing stage for checking to see whether the physiological measurement value detected by the first sensor means is representative of a critical or a noncritical physical state; and

with a display means for displaying that the physiological measurement value is representative of a critical physical state, it can be provided that the sensor signal evaluation stage is acted upon by at least one second sensor signal, which is representative of a measurement value different from the physiological measurement value detected by the first sensor means.

The processing stage is designed to check the physiological measurement value detected by the first sensor to determine if it represents a critical or a noncritical physical state under consideration of at least the second sensor signal, where in particular the short-term, medium-term, and/or long-term time change in at least one and preferably in each of the first and second sensor signals as well as possibly in additional and preferably in all sensor signals is taken into consideration, and where preferably all of the sensors or at least those intended to determine the physiological parameters are installed in the same location.

Because, according to the invention, at least one additional sensor signal is evaluated, the checking of the measurement value determined by the first sensor can be significantly improved. The possibility that a critical state such as that associated with the onset of disease will be displayed only because a single sensor, possibly even for a only a short period of time, displays an atypical value is therefore avoided. The important point here is that the second sensor is not simply another sensor identical to the first sensor, nor does the second sensor simply have a different design to provide a different way of determining the same physiological variable as the first sensor. On the contrary, a completely different variable is used in the evaluation, possibly even a different variable detected by the same sensor.

When the first sensor indicates a physical state which is critical in itself, it is possible, through consideration of this second variable, to determine easily whether or not this apparently critical physical state is plausible in the first place and possibly critical. At the same time, an earlier warning can be given when a plurality of evaluated measurements jointly suggest that a critical deviation is present.

Through the proposed joint evaluation of several sensor signals pertaining to different measurement variables it also becomes unnecessary for a physician to intervene to evaluate the individual measurements. The physician is needed only to initiate defensive or curative measures suitable for treating the physical state which has been recognized as critical.

It is also proposed according to the invention that at least two sensor signals pertaining to different measurement variables are evaluated. The idea here in particular is that the minimum of two sensor signals are selected in such a way that different measurement variables can be determined by means of at least one mathematical link between the sensor signals.

An inventive sensor means can be designed for the noninvasive, direct or indirect measurement of one of the physiological parameters from the group consisting of blood pressure, body temperature, pH, skin moisture, skin color, respiratory rate, SaO₂, SpO₂, CaO₂, LO₂, CO₂, COHb, cHb, HbMet, HbO₂, HbDe, bilirubin, glucose, heart rate, ECG, EMG, EOG, EEG, RMV (respiratory minute volume), and HMV (heart-minute volume). Noninvasive measurement from outside the body is preferred in these cases, because it causes minimal impairment to the well-being of the user of the display means.

A measurement value is determined and presented to the user when requested. All measurement values can be displayed alternatively and/or simultaneously in % saturation and/or mL/L and/or mg/L and/or mg/dL and/or g/L and/or g/dL and/or mmol/L and/or bpm and/or Hz. Thus, for example, the CaO₂ value can be displayed either as the saturation in % or in mL of oxygen per L of blood or in mL/dL. Maximum values for CaO₂ are around 200 mL/L.

The total concentration of hemoglobin (cHb) is composed of the various Hb fractions, namely, oxyhemoglobin (HbO₂), deoxygenated hemoglobin (HbDe), and the dyshemoglobins. HbO₂ transports oxygen. The dysfunctional hemoglobin derivatives such as carboxyhemoglobin (COHb), methemoglobin (HbMet) and sulfhemoglobin (HbSulf) are not able to bind oxygen. If the concentrations of these derivatives in the blood increase, they can significantly reduce the transport capacity of hemoglobin for O₂ and thus lead to hypoxemia. An increase in the dyshemoglobins can be clinically important.

After selecting the total hemoglobin concentration cHb, the user can actuate a selection function to have the value displayed in either % and/or as a quantity such as mg/L.

Alternatively and/or in addition, the cHb can be divided into the various hemoglobin fractions. In that case, alternatively or in addition, HbO₂ and/or HbDe and/or COHb and/or HbMet and/or HbSulf will be displayed. Through the use of a selection means, the Hb derivatives can be displayed alternatively and/or additionally as the percentage of cHb, for example, or as an absolute value in, for example, mg/L and/or in mmol/L.

The number of oxygen molecules available is crucial to the supply of oxygen to the cells. This number is represented by the oxygen content (CaO₂). Of less importance is the blood oxygen saturation (SaO₂), followed by the oxygen partial pressure (PaO₂). When the PaO₂ falls, O₂ is released from its bond. The CaO₂ is determined by the formula hemoglobin content (cHb)×oxygen saturation×1.34. Hb and SaO₂ are compatible over a wide range, so that lower SaO₂ values can be compensated by an increase in Hb. Under consideration of CaO₂, it is possible to allow lower PaO₂ values in many cases, if the measures necessary to increase PaO₂ represent a danger to the patient.

The oxygen concentration of the air is 21%. This corresponds to an oxygen partial pressure (pO₂) of approximately 150 mm Hg at sea level. Independently of altitude, air always contains 21% oxygen. The pO₂ value, however, falls with increasing altitude.

The organism tries to keep the oxygen supply (LO₂) for the organs and cells constant or to adapt it to the current rate of consumption. The oxygen supply (LO₂) is the product of the heart-minute volume and the oxygen content (CaO₂):
LO₂=HMV×CaO₂.

CaO₂ itself is calculated as follows:

1 mol of Hb contains 4 mol of heme iron and can bind 4 mol of O₂. 1 g of Hb binds 4×1/64,500 mol of O₂, corresponding to 0.062 mmol of O₂=1.39 mL of O₂. Because a small fraction of cHb does not have any binding activity (e.g., HbMet, COHb), the actually measured value, however, is 1.34 mL of O₂. This relationship is described by the Hüfner number (1.34). The Hufner number indicates how many mL of oxygen 1 g of Hb can bind.

CaO₂ itself is therefore calculated in simplified form as follows:
CaO₂=SaO₂×cHb×1.34.

According to the invention, the Hufner number 1.34 or a function representing the Hüfner number is stored in nonvolatile memory. In the area of the display means, either in response to a selection or automatically, the measurement values SaO₂ and cHb are linked with the Hüfner number, and the result of this link is determined preferably within one second and displayed as the CaO₂ value.

SaO₂ and Hb can be converted in relation to the CaO₂ value.

Because the changes in SaO₂, CaO₂, and cHb have a direct effect on the oxygen supply LO₂, these are the values which are preferably determined and made available as output according to the invention. A defined, clear-cut change in SaO₂ and/or CaO₂ and/or cHb can be indicated, for example, by means of an alarm. The alarm can be visual, mechanical, or acoustic. In the case of CaO₂, for example, a value of approximately 8 mL of O₂ per 100 mL of blood can be taken as an indication of an acute disorder, and a value of approximately 5 mL of O₂ per 100 mL of blood can be taken as an indication of a chronic disorder.

According to the invention, alarms are displayed when values for CaO₂ in the range of 8-5 mL of O₂ per 100 mL of blood are determined. The alarms can preferably be of different qualities and/or quantities.

Normal Hb concentrations are 155 g/L for men and 145 g/L for women. The O₂ capacity describes the maximum available amount of oxygen. The O₂ capacity is calculated by multiplying the Hb concentration by the Hüfner number.

The O₂ capacity for men: 210 mL O₂/L blood.

The O₂ capacity for women: 195 mL O₂/L blood.

Ventilation (V) enters into the calculation of the oxygen supply by way of the heart-minute volume (HMV) through the inclusion of the ventilation/perfusion quotient (V/P) as the second important control variable. The organism attempts to keep this at a constant value of approximately 0.85. Thus during physical exertion, for example, it will increase the heart-minute volume and the ventilation by the same factor.
HMV=V/P=0.85,

where V/P=the ventilation/perfusion ratio.

Instead of the HMV, therefore, we can also use the ventilation (V) as a proportional variable in the LO₂ formula.
LO₂=V×HMV×0.85.

CaO₂ and HMV are equally important with respect to LO₂.

According to the invention, a function for calculating HMV is stored in nonvolatile memory. In response to a selection, the measurement values are linked with the HMV in the area of the display means so that a value for LO₂ can be displayed. In an exemplary embodiment, HMV is calculated as follows: HMV=2 mL×heart rate×blood pressure. Alternatively, the oxygen consumption can be determined by spirometry, for example, and calculated according to Fick's formulas (Adolf Eugen Fick, 1829-1921).

The various features of novelty, which characterize the invention, are pointed out with particularity in the claims annexed to and forming part of the disclosure. For a better understanding of the invention, its operating advantages, and specific objects attained by its use, reference should be had to the drawing and descriptive matter in which there are illustrated and described preferred embodiments of the invention.

### BRIEF DESCRIPTION OF THE DRAWING

In the drawing:

FIG. 1 shows a schematic diagram of an example of a display;

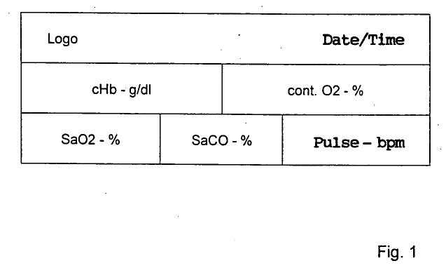

FIG. 2 shows curves representing the dependence of CaO₂, SaO₂, and cHb;

FIG. 3 shows a schematic diagram of an exemplary circuit arrangement; and

FIG. 4 shows a schematic diagram of the display means.

### DETAILED DESCRIPTION OF THE INVENTION

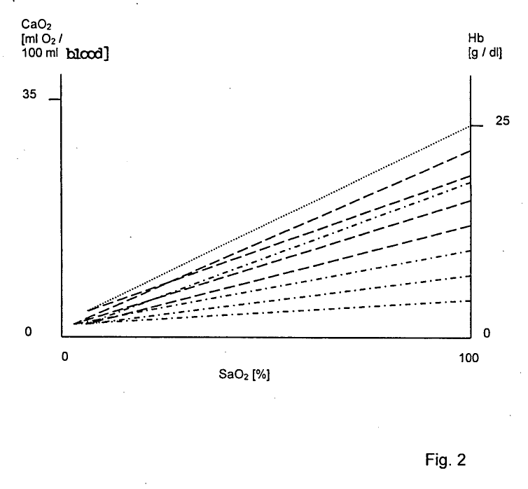

FIG. 2 shows the dependence of CaO₂, SaO₂, and cHb. The boundary regions for CaO₂ are at the points where the organism starts to change over to anaerobic metabolism in the periphery and where the hypoxia reaches the critical range. When CaO₂ is cut in half in the acute situation, the patient will in all likelihood still be in the safe range, provided that all other organs are healthy. In a hypoxemia-adapted patient who is otherwise healthy, the critical value is one-third of the normal value.

According to the invention, the user can choose to have the measurement values displayed singly, each one by itself. Alternatively and/or additionally, at least two measurement values can be displayed simultaneously and/or in alternation. The user can also select the unit of measurement to be assigned to each measurement value and thus to have either the relative value displayed in % or the absolute value in units. According to another possibility, a total amount in, for example, mmol, can be determined for each measurement value on the basis of stored calculations.

In the case of the hemoglobin concentration, the conversion is carried out as follows in the area of the device:
mmol/L=g/L×0.0621
   =g/dL×0.621
g/dL=mmol/L×1.61
g/L=mmol/L×16.1.

According to the invention, a conversion factor, e.g., 0.0621 and/or 0.621 and/or 1.61 and/or 16.1, is stored in memory. An acquired measurement value is displayed in certain units of measurement. In response to the user's selection and/or automatically, the unit is converted to an alternative unit. For this purpose, a factor stored in memory is linked with the measurement value, and the result is displayed with the alternative unit. Alternatively, the relationships can be filed as value pairs in a table.

The reference ranges for adults can be converted in a corresponding manner:

men 12.3-15.3 g/dL or 7.6-9.5 mmol/L

women 14.0-17.5 g/dL or 8.7-10.9 mmol/L.

According to the invention, a clip-like sensor is designed to be attached to an extremity of the body such as a finger or a toe or an earlobe for the duration of the measurement. According to the invention, it is also possible to use a flat sensor, which can be laid on any desired part of the body such as the forehead.

The inventive sensor means preferably has at least one optical sensor and a source of electromagnetic radiation, which can be placed on various sites on the body, as described above by way of example. An electronic evaluation circuit can be connected to the sensor.

The functional reliability of the inventive device can be increased by compensating for motion artifacts and for physiologically related measurement fluctuations.

In an especially preferred embodiment of the inventive device, a device is provided in which all essential components of the invention are housed together. The measurement time per sensor is in this case usually between 0.01 and 10 seconds. The preferred measurement range is between 40 and 100% SaO₂.

It is advantageous for the data evaluation unit to trigger an alarm only when the measured oxygen values and/or carbon monoxide values and/or oxygen content values and/or oxygen supply values and/or hemoglobin values and/or bilirubin values and/or glucose values are outside a certain range for a certain period of time.

Because short-term, one-time drops in blood oxygen saturation to saturation values of up to 80% for a period of up to 10 seconds do not represent a health risk, the adjustable limit value which can be set in the inventive device is, for example, preferably between 75 and 85% SaO₂ and even more preferably approximately 80% SaO₂. The alarm is triggered when the measurement falls below this value for a certain critical period of time, which is advantageously between 10 and 30 seconds, preferably between 15 and 20 seconds, and even more preferably approximately 15 seconds. Because, in practice, an oxygen saturation of less than 80% occurs extremely rarely for more than 15 seconds, the false-alarm rate in the inventive device is very low.

It is especially advantageous to determine not only the oxygen saturation, that is, the cHb concentration, but also the heart rate. Thus the data evaluation unit has an additional parameter which can be used to determine when an alarm is to be triggered. In addition, it is possible to provide additional measuring devices to record respiration and/or an electrocardiogram (ECG), the output signals from which are also sent to the data evaluation unit. Any additional data which may have been recorded such as the heart rate or pulse or the respiratory rate are always correlated with the primary data in subordinate fashion. Which data are to be considered primary can be specified in advance by the user or by a physician. For example, the inventive device can have a chest strap equipped with ECG electrodes. The data evaluation unit can be set up so that a drop in the heart rate and/or the respiratory rate will trigger an alarm, but it is ensured that an alarm will be triggered only if a clearly reduced oxygen value, i.e., approximately 80% SaO₂, has already been detected for longer than 10 seconds.

It is advantageous for all of the values required in an individual case to be stored continuously in a temporary memory for a period of, for example, 10 minutes. If an alarm is triggered, the data of the preceding 10 minutes are transferred from temporary memory to permanent memory in the form of a hard drive, a memory card, or the like. The data following the alarm are written to the permanent memory for a certain additional period of time (e.g., for 5 minutes), so that the time intervals before and after the alarm are available to the physician for diagnostic purposes.

It is advantageous for the inventive device also to have a display unit, on which, for example, the measured oxygen saturation values can be displayed. To prevent wide jumps, the values are first averaged over the course of, for example, at least 4 seconds or processed by the use of statistics different from those associated with the mean value and only then displayed. An acoustic alarm is given in every case, and preferably a visual warning is also displayed. Secondary alarm messages can also be provided, which, for example, can be triggered when the measurement values differ by more than an adjustable tolerance value (for example, by more than 3% SaO₂) or when the battery voltage is too low or some other system malfunction occurs. It is essential here that the secondary system alarms must be clearly different from an alarm.

It is advantageous for the inventive device also to have an interface by means of which alarm data can be transmitted directly or by modem to a local computer or to a decentralized evaluation computer in a medical center or in the office of a private physician.

For the physician, it is important to know the time at which the events were detected. This can enable the physician to establish whether risks occur more frequently at certain times of day and/or in conjunction with certain types of events. The concomitant use of a real-time clock is therefore advantageous so that the real time can also be recorded.

In the following, the invention is described in greater detail on the basis of a preferred exemplary embodiment.

Noninvasive sensors are placed on the body of the person to be monitored. For example, the sensor is placed in the area of the hand. The sensor can be, for example, attached by a strap in the area of the wrist, or it can be designed as a fingertip sensor. The measuring sensor is connected to an impedance converter and to a preamplifier.

The present invention pertains to a display means with

a sensor arrangement with at least one first sensor means for detecting physiological measurement values;

a sensor signal evaluation stage with a testing stage for checking to see whether the physiological measurement value detected by the first sensor means is representative of a critical or a noncritical physical state; and

a display means for indicating that the physiological measurement value is representative of a critical physical state.

The exemplary embodiment also comprises display means with means for detecting a physiological function of a patient and with a display device, which displays the data pertaining to the physiological function and optionally also other types of data at the same time.

According to a variant of the invention, the display unit is a monitor. This can be designed as a cathode ray tube, for example, or as an LED display, an LCD display, or as a plasma display.

It is especially advantageous for the display unit to have at least one area in which the data can be displayed graphically. The data pertaining to the physiological function can thus be displayed graphically in this area, especially in the form of a curve.

According to a variant of the invention, the other type of data pertains to an operating menu, which displays the operating functions required for an operating mode of the display means. The other type of data, however, can also be diagnostic image information. A data memory unit can be provided to store the data pertaining to the operating menu.

According to an embodiment of the invention, the display means has a control unit, to which the detecting means and the display device are connected, where the control unit controls the simultaneous presentation of the data pertaining to the physiological function and the other data on the display device.

According to an especially preferred embodiment of the invention, the display device and/or the control unit and/or the data memory are connected to a microprocessor.

When the display means is turned on, the mechanical components of the display means—to the extent that this is not already the case—are automatically reset to their default positions, and the display means is switched into an operating mode in which an operating menu with menu symbols, e.g., on the display, illustrating the various operating functions of the operating mode, are displayed. Corresponding data, including data pertaining to the control of the display means with the help of operating menus as described below, are stored in the memory unit.

The acquired data are sent to a microcontroller, which contains both the operating software for the display means and the application software. In the exemplary embodiment shown here, the microcontroller receives not only the data concerning, for example, the blood oxygen saturation but also additional information, such as information concerning the hemoglobin concentration, the carbon monoxide concentration, the oxygen content, or the heart rate or pulse and the respiratory rate. These data can be acquired by way of a chest strap equipped with electrodes and a corresponding preamplifier and received and evaluated by a microcontroller.

The display means also has a power supply, which, in the case shown here, has a power supply line and a built-in storage battery, or can be operated with batteries. The power supply has an indicator lamp, which shows whether the unit is connected to the power grid or is running on batteries. An indicator is also provided which shows the charge state and the estimated remaining operating time in battery mode. The data concerning the charge state of the battery or storage battery are also transmitted to the microcontroller.

The microcontroller controls an alarm and display device, which has an alphanumeric or graphic display, which can show all the important information concerning the device and warning messages. During normal operation, the O₂ saturation and/or the carbon monoxide saturation and/or the oxygen content and/or the hemoglobin concentration and the pulse are usually shown on the display. In addition, visual warning messages are displayed, and an acoustic alarm is given through a loudspeaker. The main alarm (i.e., the alarm which is triggered when, for example, the blood oxygen saturation is below 80% for more than 10 seconds) and some of the important system malfunction alarms (battery too low, significant measurement differences, motion artifacts, other artifacts, etc.) are presented both visually and acoustically. Other alarm functions, especially those which are based on the data supplied by the optional electrodes, are normally presented both optically and acoustically. The acoustic alarm, however, can be turned off if desired.

The measurement data are written continuously to the memory of a memory unit and, in the case of an alarm, are transferred with the actual time to a permanent memory unit. The corresponding data concerning the individual alarm events can be transferred to a diskette, to tape, or to a removable memory card (such as a Flash Card) and sent to the physician. The display means illustrated here also has an interface, via which the monitor can be connected directly to a computer for the read-out and evaluation of the data or to a modem for data transmission.

To increase user convenience, it is also possible to give the user the ability to make a preliminary selection concerning the data to be displayed and their limit values, and/or concerning the arrangement in which the data are displayed.

In an exemplary embodiment, characteristic data which give the age, sex, and weight of the user are transmitted to the display means. On that basis, sensor signal patterns which indicate a deviation from a noncritical physical state and/or allow and/or make it possible to define user-specific presettings are determined in the display means.

By means of a setting function, the user can select and adjust all of the essential functions. The desired function can preferably be set by means of a manual action. For example, the selection function can be used to preset the “sports” setting. In this case, the heart rate, the oxygen saturation, and the time in particular are displayed to the user. Within the “sports” setting, the user can enter different presets to set limit values for the heart rate and the oxygen saturation at which the corresponding alarms will be triggered.

According to another embodiment, a user can have shown to him information concerning the change in one or more parameters over time, such as in the form of a “time remaining” display. This form of display is especially suitable for the parameter carbon monoxide. Here, for example, the time-remaining display shows the time; the time-remaining display can also use different colors. As the CO saturation increases, the time-remaining display continues to move forward and thus gives the user information on how much time remains before a critical range is reached. A critical range is, for example, characterized by a change in color to red. It would also be possible for the display to blink on and off.

For COHb, a reference range of 0.4-1.6% is therefore provided according to the invention for nonsmokers and a range of 3.0-6.0% for smokers. A selection function and/or input option can therefore be used to preselect the range appropriate for a smoker or a nonsmoker.

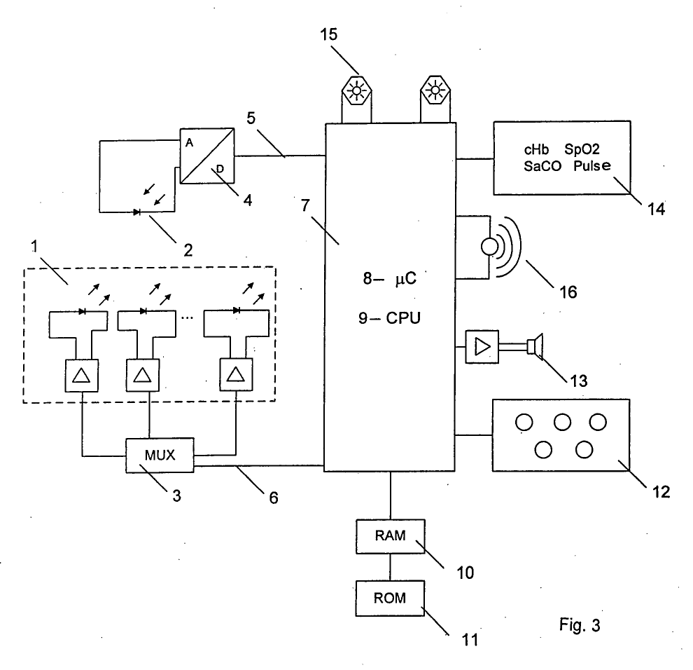

An inventive device according to FIG. 3 has a transmitter (1), in which at least one light-emitting diode LEDₐ with a first predetermined nominal wavelength λₐ is located.

Opposite the transmitter is a photodetector PD (2). Between the transmitter (1) and the photodetector PD (2), human and/or animal tissue and/or a vessel can be placed in such a way that the light emitted by the transmitter (1) passes through the tissue and/or the vessel and strikes the photodetector PD (2). The intensity of the light received by the PD is converted to an electrical variable and processed as an analog signal in the device, converted in an A/D converter, and subjected to further digital processing.

The light-emitting diodes LEDa, LEDN are connected to a multiplexer MUX (3). The control unit of the multiplexer MUX (3) controls the light-emitting diodes so that, in the case that four LEDs are connected, for example, the four LEDs are turned on and off in alternation.

The multiplexer MUX (3) has another terminal (6), which is connected to the evaluation unit (7). By means of this connection with the evaluation unit (7), the data pertaining to the power-on times of the light-emitting diodes LEDₐ to LEDₙ are transmitted. The evaluation unit has at least one microcontroller (8) or at least one CPU (9).

The output current of the photodetector PD (2) is sent to the input of a current/voltage converter (4). The current/voltage converter (4) converts the output current of the photodetector to an output voltage. In addition, the analog signal of the PD is digitized by an A/D converter of at least 8 bits and transmitted by way of an actuator to the evaluation unit (7). At least one volatile memory RAM (10) and a nonvolatile memory ROM (11) are connected to the evaluation unit (7). The nonvolatile memory (11) is in the form of, for example, an EEPROM or flash memory. An algorithm which serves to determine the measurement values is stored in the nonvolatile memory (11). An input device (12) in the form of a keyboard can be connected to the evaluation unit (7). In addition, various output devices (13, 14, 15, 16) can also be connected to the evaluation unit (7). By means of a loudspeaker (13), warning tones or voice output can be generated, for example, to inform the user or give him directions. By means of indicator lamps (15), warning signals and/or status signals can be generated. The measurement values are displayed on a display (14).

In at least one operating mode of the inventive device according to FIG. 3 shown by way of example, the tissue/vessel is exposed alternately to the light emitted by the first light-emitting diode LEDₐ and then to the light emitted by the other diodes LEDₙ where the light passing through the tissue/vessel is received by the photodetector PD and converted to a photodetector output current. The light-emitting diodes LEDₐ, LEDₙ can be operated in binary fashion, which means that at any one point the LED is either emitting light at a predetermined wavelength or not emitting any light at all. Alternatively, the LED can be driven by an analog signal of predetermined amplitude. The timing at which the LED is driven can be a function of the pulse wave phases, such as, for example, every 200 μsec.

To convert the current signal with as little noise as possible and with sufficient amplification into a voltage signal which can be used for further processing in the evaluation unit (7), it is sent to the current/voltage converter (4) and to the A/D converter. On the basis of the voltage signal, the evaluation unit (7) determines the time change in the spectral absorption of the tissue/vessel at the LED-defined wavelengths of the first and/or additional light-emitting diodes LEDₐ, LEDₙ, and by subjecting these spectral absorption values to processing and/or further processing and/or linking, it determines the measurement value of interest at the moment in question, such as the absolute or relative hemoglobin concentration cHb, the COHb concentration, the oxygen saturation SaO₂, CaO₂, or the heart rate. The measurement values for each wavelength are stored in volatile (10) and/or nonvolatile (11) memory. Then the measurement values are read out again by the evaluation unit (7) with the help of the microcontroller (8) and analyzed in the CPU (9) by means of the algorithm stored in ROM (11).

Digitized data which represent the attenuation and/or scattering of electromagnetic radiation by the tissue/vessel are processed in the CPU under program control, where a control unit retrieves the program commands from a memory and uses an arithmetic logic unit, which consists of at least one ALU, to execute the operations according to the program's instructions.

As a result, absolute and/or relative measurement values are obtained for the desired measurement value. As a function of, for example, limit values or presettings which can be defined by input on a keyboard (12), for example, the measurement value results are made available as output either electronically, visually (14, 15), and/or acoustically (13). For this purpose, the data which represent the measurement values are conditioned for an interface and made available to an interface. A protocol is preferably made available via an interface. For example, a voltage and/or a current which is essentially proportional to the measurement value is made available at the interface. Thus a digitized value representing the measurement value can be made available in a TCP/IP protocol over an Ethernet connection. For example, an SaO₂ value can be made available via a proprietary protocol at a UART interface.

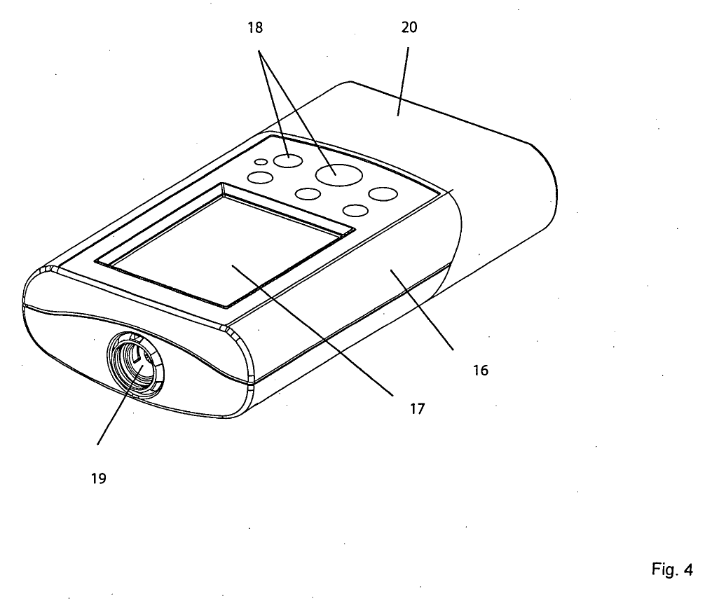

As shown in the diagram according to FIG. 4, another aspect of the invention pertains to a small, portable, handy device, which makes it possible for the user to determine several measurement values noninvasively. The device consists of a housing (17) of plastic with a recess for a display (18) and openings for operating buttons (19). The display (18) is connected electrically and mechanically to the main circuit board. An interface (20) is provided in the area of the housing. The interface (20) can be connected electrically and mechanically to the main circuit board. The interface serves to accept a sensor cable. Alternatively, the interface can be equipped as a receiving module for the wireless transmission of sensor signals. In the area of the lower shell (21), there is a socket device for an energy supply unit, such as a storage battery/batteries. In the assembled state, the bottom shell (21) and the housing are connected detachably to each other.

The dimensions of the inventive device are preferably less than 15 cm in length and less than 5 cm in depth and less than 8 cm in width. The volume of the device is preferably less than 600 ccm. To achieve small, compact dimensions and nevertheless to ensure that the device can be easily taken apart and reassembled, the device consists of no more than two circuit boards and/or fewer than 11 individual parts and/or fewer than three fastening devices.

The measurement values are displayed numerically and/or graphically, where, in the case of a graphic display, it is preferable for the time changes in the measurement values to be displayed.

The display means is designed in such a way that a measurement value can be determined either at certain times or both at certain times and continuously.

The user can choose to have the display oriented either vertically or horizontally.

The inventive display means is very small and compact and is therefore especially suitable for home monitoring. The monitor is very lightweight and transportable.

In an exemplary embodiment, the display of the display means has a scale of 10-240 units. It is preferably arranged on the outside surface to maximize legibility. It is covered by glass or plastic, for example.

The human danger zones can be identified especially effectively by means of a colored viewing field or a viewing field which changes material.

To acquire the measurement values, it is possible to use the methods described in DE 103 21 338 A1 and DE 102 13 692 A1. The methods from DE 103 21 338 A1 and DE 102 13 692 A1 are to be understood as a component of this application.

In an exemplary embodiment, electromagnetic waves, especially light, of at least two different wavelengths and/or of at least two different wavelength bands are emitted from at least one source to determine the measurement values.

The electromagnetic waves are conducted through a living and/or dead medium to be tested, preferably animal and/or human tissue.

The transmitted and/or reflected component of the electromagnetic waves is detected by the receiver system. The receiver system is able to detect different wavelengths essentially simultaneously. The receiver system is also able to record and/or to store and/or to transmit, e.g., in the form of at least one electrical pulse, the detected electromagnetic waves.

The minimum of one signal is processed by an evaluation unit through a process of signal conditioning. Independently of the original wavelength, the minimum of one signal is subjected to further processing by active and/or passive electronic components. It is preferable for the signal to be adjusted with respect to frequency and amplitude.

Digital signals which are representative of at least two different wavelengths of the original incoming radiation are analyzed by at least one CPU. It is preferable for this purpose to provide an analyzer in the area of a CPU. The signal is preferably processed in the area of the CPU. For the digital signals, at least one data memory, from which the data can be read out, is provided in the area of the CPU.

In the area of the analyzer, the following operations are carried out either alternatively, sequentially, or simultaneously:

measurement values are acquired and processed;

a pulse wave characteristic or morphology or parameters derived therefrom such as extremes, derivatives, etc., are obtained;

extinctions are determined (calculated or read out);

internal and external artifacts are cleaned up (motion, repositioning, perfusion;

parallel series of measurements are back-calculated and combined to obtain a new result;

an analog or digital signal is calculated and conditioned to control additional modules or devices.

As a result, the CPU supplies data which are representative of at least one measurement variable of the exposed medium. Artifacts are preferably cleaned up by a microprocessor, which processes the output signal of the evaluation unit in the time domain (e.g., a polynomial function) or in the Laplace domain (e.g., by means of a Fourier transformation or wavelets). The functions are selected in such a way that they are adapted to the properties of the possible artifacts.

Constants in the polynomial function are selected individually for each sensor. As part of the fabrication process, individual tests of each sensor generate a set of property constants, which pertain to the sensor errors and which are later stored in a measurement sensor EEPROM.

For the determination of the measurement values, digitized data which represent the attenuation and/or scattering of the electromagnetic radiation by tissue are linked and/or analyzed in a central unit under program control, where a control unit retrieves the commands of a program from a memory unit, and an ALU executes the operations specified by the program instructions. This ALU consists of at least one arithmetical and logical unit and makes available at least one memory unit in the area of a register.

1. A display means with at least one sensor means for detecting and transmitting physiological measurement values and one or more display means for presenting the measurement values and/or other associated data visually, acoustically, mechanically, or by some other means perceptible to the senses, comprising
a sequence control approach is implemented,
according to which the measurement value, before reaching the display, passes through a testing stage, which, on the basis of at least one measured or stored additional value, verifies, checks, or changes the physiological measurement value detected at the sensor or supplements it with additional information.

2. The display means according to claim 1, wherein the sensor means is configured to measure directly or indirectly one of the physiological measurements values from the group consisting of blood pressure, pulse, body temperature, pH, oxygen saturation, oxygen concentration, carbon monoxide concentration, carbon monoxide saturation, hemoglobin concentration, methemoglobin concentration, methemoglobin saturation, bilirubin concentration, bilirubin saturation, skin moisture, skin color, and respiratory rate.

3. The display means according to claim 1, wherein the additional value is a measured or stored value from the group consisting of blood pressure, pulse, body temperature, pH, oxygen saturation, oxygen concentration, carbon monoxide concentration, carbon monoxide saturation, hemoglobin concentration, methemoglobin concentration, methemoglobin saturation, bilirubin concentration, bilirubin saturation, skin moisture, skin color, and respiratory rate.

4. The display means according to claim 1, wherein the value or one of the additional values is determined by a time-dependent timer, especially by a clock function.

5. The display means according to claim 1, in which the sensor means is in a location separate from the rest of the device, and the measurement values are transmitted by wireless data transmission (such as by radio, infrared, or optically).

6. The display means according to claim 1, in which the display can be read optically, in either analog or digital fashion, or is designed as an acoustic signal transmitter.

7. The display means according to claim 1, wherein the testing stage is designed to evaluate the measurement values as a function of time.

8. The display means according to claim 1, wherein the testing stage generates the associated information concerning whether the physiological measurement value is representative of a defined physical state.

9. The display means according to claim 1, wherein the testing stage generates the associated information concerning whether the physiological measurement value is representative of a physical state defined as critical or noncritical.

10. The display means according to claim 1, wherein a memory stage is provided to store the measurement values and/or data derived therefrom.

11. The display means according to claim 10, wherein the testing stage is configured to test for the presence of a critical physical state under consideration of the data stored in the memory stage.

12. The display means according to claim 1, wherein the testing stage is designed to derive mathematically from several measurement values and/or measured or stored additional values the probability that a physical state defined as critical is present.

13. The display means according to claim 12, wherein the presence of a physical state defined as critical is derived from at least two separately determined probabilities.

14. The display means according to claim 1, wherein the testing stage is configured to derive the presence of a physical state defined as critical from measurements values measured over the course of a certain period of time.

15. The display means according to claim 1, wherein, as measurement values and/or additional values, the pulse is used and/or displayed in addition to the arterial oxygen saturation and/or the carbon monoxide saturation and/or values representing the hemoglobin concentration of the blood.

16. The display means according to claim 1, wherein the additional value is a measured or stored value from the group consisting of the recording of respiration and/or of an electrocardiogram (ECG) and/or of an electroencephalogram (EEG) and/or of an electromyogram (EMG).

17. The display means according to one of the preceding claims, wherein, in the testing stage, the change or the expected change in a measurement value, especially in the expected time at which a certain value is reached, is calculated.

18. The display means according to claim 1, wherein operations for converting measurement values can be carried out. in a testing stage.

19. The display means according to claim 1, wherein measurement values or measured additional values are stored in conjunction with the date and/or time of day in a memory storage unit.

20. The display means according to claim 1, in which data for use as additional values can be entered by the user or by another device.

21. The display means according to claim 1, characterized in that its display device is designed as a display, especially as an LED display, an LCD display, or a plasma display, which displays analog and/or digital signals in digital and/or analog fashion in one or more colors.

22. The display means according to claim 21, in which the way in which certain measurement values and/or data are displayed or presented can be selected individually by the user.

23. The display means according to claim 1, the display device of which displays simultaneously at least two different measurement values and/or associated information.

24. The display means according to claim 1, the display device of which displays in alternation at least two different measurement values and/or associated information.

25. The display means according to claim 1, wherein the way in which the display alternates is based on a priority calculated from the measurement values and/or the additional values.

26. The display means according to claim 1, the display device of which can output at least one warning signal.

27. The display means according to claim 1, the display device of which has at least one area in which measurement values and/or information can be displayed graphically, especially by means of symbols, analogizations, pictograms, or curves.

28. The display means according to claim 1, the display device of which displays data which are representative of the absolute concentration of HbO₂, COHb, cHb, HbMet, deoxyHb, bilirubin, glucose, or heart rate.

29. The display means according to claim 1, the display device of which displays data which are representative of a relative percentage of at least one of the following variables: HbO₂, COHb, cHb, HbMet, deoxyHb, bilirubin, glucose, or heart rate.

30. The display means according to claim 1, the display device of which can output alternatively especially two or more of the units g/L, g/dL, mg/dL, mmol/L, %, bpm in relation to one variable.

31. The display means according to claim 10, wherein the units are converted by means of a stored factor.

32. The display means according to claim 10, wherein the units are correlated with each other and stored in a table in readable form.

33. The display means according to claim 1, the display device of which can be attached in the area of a user.

34. The display means according to claim 1, the display device of which displays, optionally or alternatively or additionally, the date and/or time of day.

35. The display means according to claim 1, the display device of which is preferably located in the area of a plastic housing.

36. The display means according to claim 1, the display device of which is located on the top surface of the device.

37. The display means according to claim 1, the display device of which has a separate operating element for the display device.

38. The display means according to claim 1, the display device of which has an integral operating element for the display device.

39. The display means according to claim 1, wherein the operating element requires only one actuation for an output process.

40. The display means according to claim 1, wherein the operating element is protected especially against unintentional or incorrect use.

41. The display means according to claim 1, wherein an operating element is provided to reset the device to a defined standard state.

42. The display means according to claim 1, the display device of which outputs not only measurement values but also additional information simultaneously, namely, defined, physiologically relevant limit values, especially by analog presentation.

43. The display means according to claim 1, the display device of which allows both analog and digital presentation alternatively, successively, or adjacent to each other.

44. The display means according to claim 1, the display device of which has at least two display fields for different measurement values and/or associated information, the design or division of which is changed automatically as a function of the defined importance of the displayed measurement values or information.

45. A process for noninvasive determination of the oxygen content (CaO₂) of human and/or animal tissue, comprising emitting electromagnetic radiation with wavelengths in the range of 400-1,800 nanometers from at least one source; conducting the radiation through a vessel/tissue; detecting by means of a detector the radiation after it has passed through the tissue and/or after it has been scattered by the vessel/tissue; and digitizing the data, further comprising determining the oxygen saturation (SaO₂) of the blood by means of at least one mathematical link of the data, and determining the concentration of hemoglobin in the blood (cHb) by means of at least one additional mathematical link of the data.

46. The process according to claim 45, wherein the oxygen content (CaO₂) is acquired by means of a link of SaO₂ with cHb.
